(19) United States
(12) Patent Application Publication
(10) Pub. No.: US 2015/0080770 A1
(43) Pub. Date: Mar. 19, 2015

(54) DIFFERENTIATION OF STEM CELLS WITH NANOPARTICLES

(71) Applicant: The Research Foundation for The State University of New York, Albany, NY (US)

(72) Inventors: Balaji SITHARAMAN, Coram, NY (US); Jon LONGTIN, Port Jefferson, NY (US)

(21) Appl. No.: 14/554,433

(22) Filed: Nov. 26, 2014

Related U.S. Application Data
(63) Continuation of application No. 13/132,060, filed on Dec. 16, 2011, now abandoned.
(60) Provisional application No. 61/118,937, filed on Dec. 1, 2008.

(51) Int. Cl. A61N 7/00

(52) U.S. Cl. CPC A61N 7/00
USPC 601/2

(57) ABSTRACT

This invention relates to stimulation of stem cells and other progenitors of differentiated cells using nanoparticles and electromagnetic stimulation. The invention provides a method for differentiating mesenchymal stem cells (MSCs) towards osteoblasts and other connective tissue. The invention provides osteoinductive materials useful for bone regeneration and reconstruction in treatment of bone trauma and bone related diseases, and to correct birth defects. The invention also provides for reduced adipogenesis.

### CROSS REFERENCE TO RELATED APPLICATIONS

This application claims priority to U.S. Application No. 61/118,937, filed Dec. 1, 2008, which is incorporated herein by reference in its entirety.

### FIELD OF THE INVENTION

This invention relates to stimulation of stem cells and other progenitors of differentiated cells using nanoparticles and electromagnetic stimulation. The invention provides a method for differentiating mesenchymal stem cells (MSCs) towards osteoblasts and other connective tissue. The method is useful for bone regeneration and reconstruction in treatment of bone trauma and bone related diseases, and to correct birth defects. The invention also provides for decreased levels of adipogenesis.

### BACKGROUND OF THE INVENTION

The mechanical (acoustic) stimulation of cells has been shown to increase bone regeneration and decrease adipogenesis, while electromagnetic stimulation has been shown to enhance bone and neuronal regeneration.

Nanobiomaterials have promising applications in the biomedical field in areas including tissue engineering, drug delivery, biosensors, and bioimaging. For example, gold nanoparticles (GNPs) and single-walled carbon nanotubes (SWNTs) have potential for diagnostic and therapeutic applications because they are easily conjugated with biological molecules, and have useful mechanical, electrical, and physical properties. Previous studies show that both SWNTs and GNPs absorb radiofrequency electromagnetic radiation and light in the near infrared (NIR). This can lead to a localized increase in temperature, such as in tumor tissue where the nanoparticles can be located, which will cause tumor destruction due to induced hyperthermia. When the electromagnetic radiation is in a frequency range that is poorly absorbed by healthy tissue (such as laser radiation in the NIR region), thermal ablation occurs only where nanoparticles are localized.

Lasers are used in many biomedical applications such as bioimaging, hair and skin lesion removal, wound healing, ablation and much more. What these all have in common is the interaction of the laser light with a biological system. Pulse lasers and continuous lasers have different effects and are specific for the medical application. When a low energy nanosecond pulsed laser transmits non-ionizing electromagnetic energy onto an absorbing surface, this gives rise to a thermoelastic expansion leading to a wideband ultrasonic emission. This process is known as the photoacoustic effect. The photoacoustic effect dates back to Alexander Graham Bell and has recently been used for bioimaging applications. In this regard, little is known about the effects on tissue of nanoparticles stimulated with a pulsed electromagnetic radiation.

### SUMMARY OF THE INVENTION

The invention provides a method of stimulating stem cells and other progenitor cells, such as, for example, marrow stromal cells, in which nanoparticles, including carbon or gold nanoparticles, absorb electromagnetic radiation and transmit mechanical energy to the cells. For example, nanoparticles that absorb light or radiofrequency electromagnetic radiation at GHz or near GHz frequencies are employed to stimulate stem cells in culture or in situ.

The instant invention relates to stimulation of stem cells and progenitor cells. The invention relates to stem cells at various stages of differentiation, and includes, for example, totipotent, pluripotent, multipotent, and unipotent stem cells. According to the invention, a stem cell is stimulated to proliferate and/or differentiate by culturing the cell in culture media in the presence of nanoparticles, and subjecting the cell culture to electromagnetic radiation to induce mechanical resonance of the particles. Nanoparticles of the invention include, but are not limited to, carbon nanoparticles, including but not limited to carbon nanotubes, single walled carbon nanotubes (SWNTs), graphene nanoparticles, and graphite nanoparticles. In another embodiment of the invention, the nanoparticles are metal nanoparticles, such as gold nanoparticles (GNPs). The electromagnetic and optical absorbance properties of the nanoparticles result from the composition of the nanoparticles themselves or from moieties linked to the nanoparticles.

According to the invention, a stem cell is cultured in the presence of nanoparticles. In one embodiment, the nanoparticles are in contact with the culture media, and may be in direct contact with the cells. In another embodiment, the nanoparticles are in the culture media, but separated from the stem cell, for example, by a membrane. In yet another embodiment, the nanoparticles are adjacent to, but not in the culture media and cells. For example, the SWCNTs can be embedded in or in contact with the external surface of a container of the culture media.

As used herein, the term “about” when used in conjunction with a physical measurement, such as length, frequency, wavelength and the like refers to any number within 1, 5, 10, or 20% of the referenced number.

Nanoparticles of the invention convert electromagnetic radiation to acoustic energy. The size of the nanoparticles used according to the invention can be homogenous or variable. For example, in an embodiment of the invention, SWNTs range from about 10 nm to about 200 nm. In an embodiment of the invention, the size of the SWNTs is about 1-2 nm in diameter. Similarly, metal nanoparticles, such as GNPs are used. In one embodiment, the GNPs are spherical. In another embodiment of the invention, the GNPs are rod shaped. In an embodiment of the invention, the GNPs are from about 10 nm to about 50 nm in diameter.

According to the invention, the nanoparticles are subject to electromagnetic radiation at a frequency and intensity that results in stimulation of stem cells or other progenitor cells. In one embodiment of the invention, the frequency of electromagnetic radiation is from about 10 MHz to about 10 GHz. In another embodiment of the invention, the frequency of electromagnetic radiation is from about 500 MHz to about 5 GHz. In another embodiment of the invention, the frequency of electromagnetic radiation is about 3 GHz. In yet another embodiment, the frequency of electromagnetic radiation is that of a medically useful RF source such as an MRI scanner. Also according to the invention, ultraviolet light, visible light, or infrared radiation, such as near infrared radiation can be used. In an embodiment of the invention, the wavelength of electromagnetic radiation is from about 100 nm to about 2000 nm. In another embodiment of the invention, the wavelength of electromagnetic radiation is from about 250 nm to about 1000 nm. In another embodiment, the wavelength of electromagnetic radiation is that of a medically light source, including but not limited to, about 532 nm, about 633 nm, about 764 nm, or about 1064 nm.

The electromagnetic radiation can be constant of be pulsed. In an embodiment of the invention, the pulse frequency is from about 5 Hz to about 500 Hz. In one embodiment, 3 GHz electromagnetic radiation is pulsed with a repetition rate of about 100 Hz and a pulse duration of about 0.5 μs. In another embodiment of the invention, 532 nm electromagnetic radiation is pulsed with a repetition rate of about 10 pulses per second and a pulse duration of about 200 ns.

The invention applies to a variety of stem cells of various types and stages of differentiation and from a variety of sources, and cultured in media that promotes differentiation towards a particular cell type. In one such non-limiting embodiment of the invention, the stem cell is a mesenchymal stem cell. In a particular embodiment, the stem cell is a bone marrow stromal cell. In another embodiment, the culture media is osteogenic. In another embodiment, the culture media is chondrogenic.

Accordingly, the invention also provides a method of obtaining a differentiated cell by culturing a progenitor cell in culture media in the presence of nanoparticles and subjecting the cultured cell to electromagnetic radiation that induces mechanical resonance of the nanoparticles. In one embodiment, the differentiated cell is an osteoblast. In another embodiment, the differentiated cell is a chondrocyte. In another embodiment, the differentiated cell is a muscle cell. In yet another embodiment, the differentiated cell is a nerve cell. According to the invention, the progenitor cell can be, for example, a mesenchymal stem cell such as a bone marrow stromal cell. In another embodiment, the progenitor cell is an embryonic (ES) cell.

The invention also provides a method for stimulating growth or regeneration of bone, cartilage, muscle, or nervous tissue. In certain embodiments, progenitor cells in tissue are stimulated directly using nanoparticles and electromagnetic radiation to stimulate the nanoparticles. In another embodiment, a matrix, such as an osteogenic matrix comprising nanoparticles and bone forming cells is treated with electromagnetic radiation that induces mechanical resonance of the nanoparticles. In one embodiment, the osteogenic matrix is stimulated in vitro. In another embodiment of the invention, the osteogenic matrix is stimulated in situ. In another embodiment, the matrix is a chondrogenic matrix. In another embodiment, progenitor cells are incorporated into an implant or prosthesis and stimulated in situ.

The invention also provides stimulated stem cells and progenitor cells and differentiated cells. In one embodiment, the stimulated stem cells are mesenchymal stem cells. In another embodiment, stimulated stem cells are stimulated ES cells. According to the invention, the differentiated cells include, but are not limited to, osteocytes, chondrocytes, neural cells, muscle cells, and cardiac myocytes. In an embodiment of the invention, the stimulated stem cells or differentiated cells are used to identify and/or isolate biological compounds, including but not limited to proteins and nucleic acids characteristic of the stimulated or differentiated state of the cells. Such compounds are useful, for example, as markers of differentiation, and as targets for antibodies and other agents.

The invention also provides a composition for stimulating and/or differentiating stem cells or progenitor cells. The compositions are suitable for cell growth and contain nanoparticles. In one embodiment, the composition is a polymer comprising nanoparticles dispersed within. In an embodiment of the invention, the composition is a film, which may be free standing or coated on a support. In another embodiment, the composition is a porous structure composed of a polymer, which may be biodegradable, comprising nanoparticles dispersed within. In one embodiment of the invention, the polymer is poly(D,L-lactic-co-glycolic acid) (PLGA). In certain embodiments, the lactic acid-glycolic acid ration is 50:50, 65:35, or 75:25. In another embodiment, the polymer is polylactide (PLA).

The invention further provides kits for differentiating stem cells. The kits comprise stem cells and nanoparticles for stimulating the stem cells. The nanoparticles can be provided in containers separate from the stem cells or embedded in containers for culturing the stem cells. In another embodiment, the kits contain nanoparticles incorporated into a support, such as a film or a scaffold on (or within) which stem cells or progenitor cells are propagated and/or differentiated. Optionally, the kits further contain media formulations selected to promote differentiation to osteocytes, chondrocytes, or other differentiated cell types.

### BRIEF DESCRIPTION OF THE FIGURES

FIG. 1 shows calcium levels of MSC samples stimulated with RF that were in direct and indirect contact with SWNTs. *Significant difference between RF and non RF, p<0.05; ** Significant difference between SWNT and no SWNT cultures for the same medium condition, p<0.05.

FIG. 2 shows changes in osteoblast characteristics at 4, 9 and 16 days after initiation of stimulation by a 532 nm Nd:Yag laser and gold nanoparticles. Culture conditions: SWNT in media=SWNTs incubated with cells with photoacoustic (PA) stimulation; gold=gold nanoparticles coated to outside bottom of tissue culture well with PA stimulation; SWNT=SWNT nanoparticles coated to outside bottom of tissue culture well with PA stimulation; light=no nanoparticles with PA stimulation; control 1=no nanoparticles, no PA stimulation; control 2=SWNT nanoparticles incubated with cell, no PA stimulation; control 3=osteogenic supplement, no nanoparticles, no PA stimulation. Panel A: Calcium expression is a late stage marker for osteogenic activity. Panel B: ALP expression is an early stage marker for osteogenic activity. Panel C: Cellularity of the MSCs. Panel D: OPN protein is synthesized by cells during bone development and secreted into the extracellular fluid.

FIG. 3 shows the time course of OPN synthesis. Culture conditions: gold=gold nanoparticles coated to outside bottom of tissue culture well with PA stimulation; CNT=SWNT nanoparticles coated to outside bottom of tissue culture well with PA stimulation; light=no nanoparticles with PA stimulation; control 1=no nanoparticles, no PA stimulation.

FIG. 4 compares calcium content for cells in wells that were stimulated directly (wells in the path of the laser during PA stimulation) and indirectly (adjacent well not in the path of the laser).

FIG. 5 depicts an experimental setup for photoacoustically stimulating cells. The view of the well shows the incident laser pulse perpendicular to the cellular surface, carbon nanotube layer, and cell media. Not depicted is the laser (typically an Nd:YLF laser) or apparatus for adjusting incidence of the laser.

In FIG. 6, cellularity was quantified for each group after 4, 9, and 15 days in culture. The non-stimulated samples include cells cultured on a glass slide (Light Control), a PLGA film (PLGA Control), a PLGA film incorporated with SWNTs (PLGA-SWNT Control), and a glass slide containing osteogenic supplemented media in the cell culture well (Dex). The stimulated samples were exposed to the laser for 10 minutes a day and they include cells cultured on a glass slide (Light), a PLGA film (PLGA), and a PLGA film incorporated with SWNTs (PLGA-SWNT). At 9 days in culture, there was a significant difference between stimulated samples and their non-PA stimulated counterparts (*p<0.05).

FIG. 7 depicts a quantitative analysis for alkaline phosphatase (ALP) expression for non-stimulated and stimulated cells with and without PA stimulation after 4, 9, and 15 days in culture. The non-stimulated samples include cells cultured on a glass slide (Light Control), a PLGA film (PLGA Control), a PLGA film incorporated with SWNTs (PLGA-SWNT Control), and a glass slide containing osteogenic supplemented media in the cell culture well (Dex). The stimulated samples were exposed to the laser for 10 minutes a day and they include cells cultured on a glass slide (Light), a PLGA film (PLGA), and a PLGA film incorporated with SWNTs (PLGA-SWNT). At all time points, there was a significant difference between the stimulated samples and their non-stimulated counterparts (*p<0.05). After 9 and 15 days in culture, there was also a significant difference between PA stimulated samples cultured on PLGA-SWNT films in comparison to all other groups (**p<0.05).

FIG. 8 depicts a quantitative analysis of calcium matrix deposition for stimulated and non-stimulated cells with and without PA stimulation after 4, 9, and 15 days in culture. The non-stimulated samples include cells cultured on a glass slide (Light Control), a PLGA film (PLGA Control), a PLGA film incorporated with SWNTs (PLGA-SWNT Control), and a glass slide containing osteogenic supplemented media in the cell culture well (Dex). The stimulated samples were exposed to the laser for 10 minutes a day and they include cells cultured on a glass slide (Light), a PLGA film (PLGA), and a PLGA film incorporated with SWNTs (PLGA-SWNT). At all time points, there was a significantly higher level of calcium for the stimulated samples compared to their non-stimulated counterparts (*p<0.05). At all time points in culture, there was also a significantly greater amount of calcium for stimulated samples cultured on the PLGA-SWNT film in comparison to all other groups (**p<0.05).

FIG. 9 shows osteopontin concentrations in media from MSC undergoing PA stimulation for 10 minutes per day. The non-stimulated samples include cells cultured on a glass slide (Light Control), a PLGA film (PLGA Control), a PLGA film incorporated with SWNTs (PLGA-SWNT Control), and a glass slide containing osteogenic supplemented media in the cell culture well (Dex). The stimulated samples were exposed to the laser for 10 minutes a day and they include cells cultured on a glass slide (Light), a PLGA film (PLGA), and a PLGA film incorporated with SWNTs (PLGA-SWNT). Osteopontin expression was consistently higher in the PA stimulated groups, with the PLGA-SWNT group having the greatest expression.

FIG. 10 shows Alizarin red optical images from left to right of PA stimulated PLGA-SWNT (PLGA-SWNT), PA stimulated PLGA (PLGA), osteogenic supplemented control (Dex), and PA stimulated direct light (Light). Circle diameters correspond to 15 mm.

### DETAILED DESCRIPTION OF THE INVENTION

The invention provides a method for stimulating stem cells and progenitor cells by treating the cells with nanoparticles and non-ionizing electromagnetic radiation that induces acoustic vibrations in the nanoparticles. In certain embodiments of the invention, the stem cells or progenitor cells are grown in culture and treated. In other embodiments, stems cells or progenitor cells are stimulated in situ.

The nanoparticles can be of various size and composition, so long as they can be excited to radiate acoustic (mechanical) energy in response to irradiation with an electromagnetic source. The electromagnetic absorbance properties of the nanoparticles result from the composition of the nanoparticles themselves or from moieties linked to the nanoparticles. The nanoparticles can be composed of a variety of substances, including metals such as gold, silver, and titanium. Nanoparticles of the invention further include carbon nanoparticles, including but not limited to carbon nanotubes, single walled carbon nanotubes (SWNTs), graphene nanoparticles, and graphite nanoparticles. Nanoparticles of the invention also include nanotubes composed of, for example, boron nitride. Also, as mentioned, desired absorbance properties can be obtained by linking sensitizing dyes to the nanoparticles. In certain embodiments of the invention, the nanoparticles are selected to be excited at wavelengths at which human tissue is relatively transparent. In one embodiment exemplified herein, the nanoparticles are gold nanoparticles. In another embodiment exemplified herein, the nanoparticles are single walled carbon nanotubes. The nanoparticles of the invention can be relatively homogenous in size and shape, or be variable. The nanoparticles can be conjugated to other moieties, such as, for example, targeting moieties to immobilize the nanoparticles at a selected location in the body, or moieties that enhance interactions with particular cell types. In an embodiment of the invention, nanoparticles composed of or linked to, for example, bisphosphonate, are used.

According to the invention, electromagnetic radiation over a wide range of frequencies can be used to induce acoustic vibrations in the nanoparticles. In one embodiment of the invention, high frequency (HF) electromagnetic radiation (about 3 MHz to about 30 MHz) is selected. In another embodiment of the invention, very high frequency (VHF) electromagnetic radiation (about 30 MHz to about 300 MHz) is selected. In another embodiment of the invention, ultra high frequency (UHF) electromagnetic radiation (about 300 MHz to about 3 GHz) is selected. In another embodiment of the invention, super high frequency (SHF) electromagnetic radiation (about 3 GHz to about 30 GHz) is selected. In another embodiment of the invention, extremely high frequency (EHF) electromagnetic radiation (about 30 GHz (1 cm) to about 300 GHz (1 mm)) is selected. In other embodiments, infrared radiation is selected such as, for example, far infrared (about 300 GHz (1 mm) to about 30 THz (10 μm)), mid-infrared (about 30 THz (10 μm) to about 120 THz (2.5 μm)), or near infrared (about 120 THz (2.5 μM) to about 400 THz (750 nm)). In other embodiments, electromagnetic radiation in the visible region (about 400 nm to about 700 nm) or in the ultraviolet region (about 50 nm to about 400 nm) is selected. In certain embodiments, the electromagnetic radiation is coherent (e.g., generated by a laser). As mentioned, it is often useful to select frequencies or wavelengths to which the human body is relatively transparent (i.e., frequencies up to near infrared). In this regard, methods of the invention can often be facilitated by using electromagnetic fields generated by equipment already in use in hospitals and health care facilities. For example, the RF range around 40-50 MHz is used in nuclear magnetic resonance (NMR) and typical magnetic resonance imaging (MRI) uses frequencies from under 1 MHz up to about 400 MHz. Some examples include 13.56 MHz, 42.58 MHz (1-T scanner) and 63.86 MHz (1.5-T scanner). In one example disclosed herein, SWNTs were irradiated with SHF electromagnetic radiation (about 3 GHz). Infrared, visible, and ultraviolet light sources can also be used for stimulation. Commonly used wavelengths include, but are not limited to, 532 nm, 633 nm, 764 nm, and 1064 nm. In another example, gold nanoparticles were illuminated with coherent visible light (532 nm).

According to the invention, the radiation is pulsed in a manner that results in acoustic (mechanical) vibrations and avoids heating of cells or tissues. For example, in the electromagnetic radiation is pulsed at a frequency from about 5 to about 500 Hz, or from about 10 Hz to about 100 Hz. In one example, 3 GHz radiation was pulsed at 100 pulses/sec. with a pulse duration of 0.5 μs. In another example, a 532 nm laser was pulsed at a rate of 10 pulses/sec. with a pulse duration of 200 ns. Heating can also be prevented by limiting the intensity of the electromagnetic radiation.

According to the invention, the nanoparticles are disposed such that the mechanical or acoustic vibrations induced in the particles are transmitted to the cells being treated. For example, the nanoparticles can be included in a culture (i.e., in the culture media) of stem cells or progenitor cells, or be separated from the culture, for example by the vessel which contains the cell culture, as long as acoustic vibrations can be transmitted to the cells. For example, the nanoparticles can be embedded in, immobilized on, or otherwise in contact with the inner or outer surface of a tissue culture container, slide, or other cell culture vessel or device. In another example, nanoparticles are embedded in or immobilized on a device that can be contacted with a tissue or other collection of cells containing cells to be stimulated. In yet another example, the nanoparticles are contained in or immobilized to a scaffold whereupon stem cells or progenitor cells are stimulated to propagate and or differentiate.

Of particular interest are mesenchymal stem cells (MSCs) which can differentiate, in vitro or in vivo, into a variety of connective tissue cells or progenitor cells, including, but not limited to, including mesodermal (osteoblasts, chondrocytes, tenocytes, myocytes, and adipocytes), ectodermal (neurons, astrocytes) and endodermal (hepatocytes) derived lineages. The terms “mesenchymal stem cell” and “marrow stromal cell” are often used interchangeably, so it is important to note that MSCs encompass multipotent cells from sources other than marrow, including, but not limited to, muscle, dental pulp, cartilage, synovium, synovial fluid, tendons, hepatic tissue, adipose tissue, umbilical cord, and blood, including cord blood. Also of interest are embryonic stem (ES) cells, which can be differentiated into all cell types.

According to the invention, nanoparticles and stimulatory electromagnetic radiation can be employed not only in tissue culture, but wherever it is desired to stimulate growth and/or repair of connective tissue, muscle, or nervous tissue. According to the invention, nanoparticles and stimulatory electromagnetic radiation can be used to stimulate stem cells, such as MSCs, in a host. The stem cells can be cells already present at a particular location, or implanted or injected cells.

In certain embodiments, the stem cells are implanted as part of a tissue or prosthesis. For example, nanoparticles for stimulation of stem cells are used in preparation (i.e., incorporated in) or treatment of structures that are destined for insertion or implantation into a host.

One example of such a structure is a matrix for bone or cartilage growth or regeneration. Examples include, but are not limited to a demineralized bone matrix (e.g., composed primarily of collagen and non-collagenous proteins), devitalized cartilage matrix, or other artificial matrix for bone or cartilage regeneration. Other porous scaffolds (ceramics, metals, polymers, and nano-reinforced) are osteoconductive, and promote bone ingrowth, with osteoinductive properties provided by incorporation of peptides, hydroxyapetite, or growth factors and cytokines known to influence bone cells. In one embodiment, a factor that promotes osteogenesis is linked to a SWNT that is incorporated into the scaffold. For example, apatite can be attached to SWNTs.

In one embodiment, collagen, particularly collagen type II, is used to promote chondrogenic differentiation.

Thus, the matrices can include bone- or cartilage-specific matrix components and are populated with bone or cartilage progenitor cells, which are stimulated according to the invention pre- and/or post-implantation when the matrix is subject to electromagnetic radiation.

In a non-limiting example disclosed below, SWNTs are incorporated into poly(D,L-lactic-co-glycolic acid; 50:50) (PLGA) polymer films, and the effect of pulse-laser induced photoacoustic (PA) stimulation of MSCs seeded on PLGA/SWNT films in demonstrated. PLGA is representative of polymers used in the fabrication of tissue engineering scaffolds, and is biocompatible, biodegradable, and FDA approved for clinical use. Useful polymers further include, but are not limited to, polylactide (PLA), and PLGAs having a different lactic to glycolic acid ratio (e.g., 65:35, 75:25). Nanoparticles other than SWNTs may be similarly incorporated into scaffolds. For example, PLGA can be dissolved (e.g., in chloroform) or melted and nanoparticles dispersed in the solution (e.g., by sonication). The dispersals can be formed into 2D and 3D structures such as by coating onto a surface or preparing as a porous form or fibers. The dispersals can be fabricated, for example, by solvent casting, melt processing, extrusion, injection and compression molding, and spray drying.

SWNT mediated PA stimulation of MSCs is demonstrated to result in enhanced differentiation of the MSCs towards osteoblastic lineages, as shown by quantitative analysis of known indicators for cell proliferation (cellular DNA analysis), and differentiation (production of alkaline phosphatase (ALP), deposition of a calcified matrix (Ca content analysis), and osteopontin (OPN) expression). Alizarin Red staining is an example of a qualitative measure of calcium deposition in the extracellular matrix and confirms the calcium content analysis.

The method of the invention is also applied to the manufacture and use of a medical implants, such as an orthopedic or a dental implant. The implant can be a metal implant, such as an artificial hip, knee, or shoulder, to which bone must meld. Other examples include dental implants. The implants are prepared with carbon nanotubes or other nanoparticles attached at surfaces that are to be fused to bone, providing an improved surface that enhances growth of bone forming cells. The implant can also be made of a composite material such as a fiber composite. For example, along with carbon fiber and fiberglass composites, orthopedic implants can be made from composite materials strengthened by the addition of carbon nanotubes. Nanotube-like structures composed of other substances, such as boron can also be used. As provided above, the carbon nanotubes can optionally be modified with apatite. The implants can be implanted directly, or incubated with osteoblasts from the recipient prior to implantation. The implants are subjected to electromagnetic radiation according to the invention prior to and/or after implantation. Preincubation with osteoblasts and stimulation of osteoblasts according to the invention is particularly advantageous if the implants are opaque to electromagnetic radiation in a way that would block irradiation in situ. Nevertheless, preincubation and electromagnetic stimulation is useful even where the implants are transparent to the stimulatory electromagnetic radiation.

When implanted or injected, stem cell development is often governed by the site of implantation or the site in the body to which the stem cells home. According to the invention, differentiation of stem cells and progenitor cells can also be directed in vitro by selection of media components and/or matrix components. For example, cytokines and growth factors that promote osteogenic differentiation include various isoforms of bone morphogenetic protein (BMP) such as BMP-2, -6, and -9, interleukin-6 (IL-6), growth hormone, and others. (See, e.g., Heng et al., 2004, J. Bone Min. Res. 19, 1379-94). Cytokines and growth factors that promote chondrogenesis include various isoforms of TGF-β and bone morphogenetic protein, activin, FGF, and other members of the TGF-β superfamily. Chemical factors that promote osteogenesis and chondrogenesis to include prostaglandin E2, dexamethasone. Osteogenesis or chondrogenesis can also be favored by selection of extracellular matrix (ECM) material. For example, chondrogenesis is favored by naturally occurring or synthetic cartilage extracellular matrix (ECM). Such an ECM can comprise collagenous proteins such as collagen type II, proteoglycans such as aggrecan, other proteins, and hyaluronan. (See, e.g., Heng et al., 2004, Stem Cells 22, 1152-67). Phenotypic markers expressed by cells of the various lineages are well known in the art.

Nanoparticles and stimulatory electromagnetic radiation are also employed to inhibit differentiation of adipocyte progenitor cells to adipocytes. As used herein, inhibition of differentiation to adipocytes means that differentiation is reduced, but not necessarily prevented entirely. In an embodiment of the invention, to inhibit differentiation to adipocytes, nanoparticles are placed in the vicinity of the adipocyte progenitors. For example, the nanoparticles are injected into fat tissue, or incorporated into a matrix that is inserted into fat tissue. Alternatively, the nanoparticles can be applied on the skin, for example in a cream or ointment, or embedded in a film, patch or other covering that is applied near the fat tissue. Electromagnetic radiation is then applied to induce mechanical stimulation of the tissue by the nanoparticles.

The invention also provides a means for investigating stem cell or progenitor cell differentiation. For example, the invention provides a method of identifying a cellular component that is differentially expressed in a stimulated stem cell. A stem or progenitor cell of interest is cultured in the presence of nanoparticles which are and subjected to electromagnetic radiation to induce photoacoustic (mechanical) vibration of the nanoparticles. Test cells thus stimulated are compared to a control cells (cultured under the same conditions but without exposure to electromagnetic radiation) and evaluated with changes in cellular components and or cellular phenotypes, including but not limited to growth characteristics and differentiation markers.

It is to be understood and expected that variations in the principles of invention herein disclosed may be made by one skilled in the art and it is intended that such modifications are to be included within the scope of the present invention. The following examples only illustrate particular ways to use the novel technique of the invention, and should not be construed to limit the invention.

### EXAMPLES

### Example 1

### Stimulation with SWNTs and RF

Mouse bone marrow stromal cells (ATCC crl-12424) were seeded at 20,000 cells per well in 24 well plates and cultured in non osteogenic media containing Dulbecco's modified essential medium, 10% fetal bovine serum, and 1% penicillin/streptomycin, or osteogenic media (OM) which also contained 10⁻⁸ M dexamethasone, 10 mM β-glycerophosphate, and 5 μg/ml L-ascorbic acid. For the groups “SWNT in OM+RF” (radiofrequency) and “SWNT in OM”, the SWNTs were directly incubated with the MSCs. To resolve the contribution of mechanical acoustic waves generated from the SWNTs on the differentiation of the MSCs, other groups were created which avoided direct contact of the SWNTs with cells. Using 12 mm circular glass cover-slips, 2 μg of SWNTs were secured below (on the outside) the surface of half of the cell plates containing OM and non-OM and these SWNTs had no direct contact with the cells.

Every group was stimulated with RF at 3 GHz, with a 0.5 μs pulse duration, and 100 Hz pulse rate for 15 minutes a day for 5, 12, and 18 days to compare to the non stimulated counterpart. The differentiation of MSCs and their non-stimulated controls was then determined by analysis of known indicators of the osteoblast phenotype including cell proliferation, production of alkaline phosphatase, and deposition of a calcified extracellular matrix. Statistical analysis was done using a one-way ANOVA. The data is presented as means±standard deviation for n=6 for each sample group. If the ANOVA test detected significance, Tukey's ‘Honestly Significantly Different’ (HSD) multiple comparison test was then used to determine the effects of the parameters examined. All comparisons were conducted at a 95% confidence interval (p<0.05).

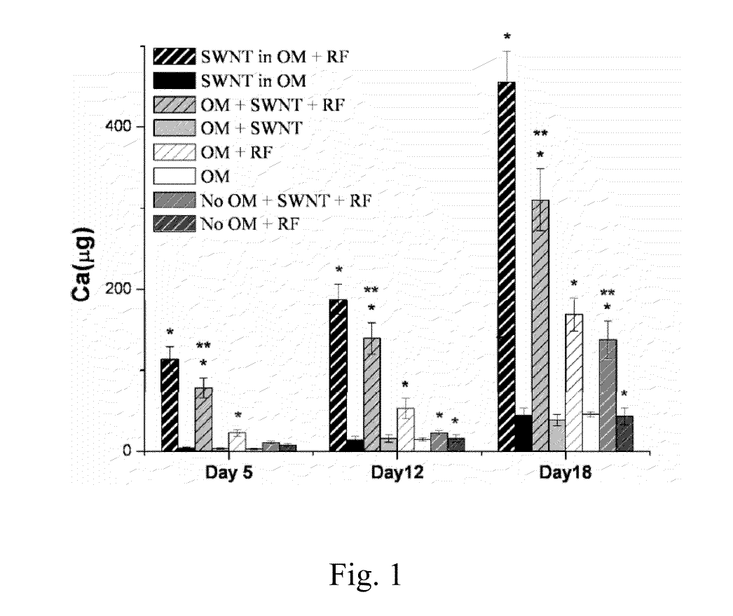

FIG. 1 shows the calcium content of the various groups after 5, 12, and 18 days of culture. The results clearly show increased levels of calcium in samples induced by RF and SWNTs, with up to a 10-fold increase in calcium content compared to the controls. The groups without RF stimulation and no SWNTs had negligible changes in calcium.

The results demonstrate the effect of thermoacoustic (TA) stimulation on MSC differentiation, and show that the presence of SWNTs enhances this effect. The results highlight the promise of TA stimulation for tissue regeneration as well as the potential of SWNTs to improve the bioactivity of tissue engineering scaffolds in the presence of TA stimulation.

### Example 2

### Stimulation with Nanoparticles and Laser

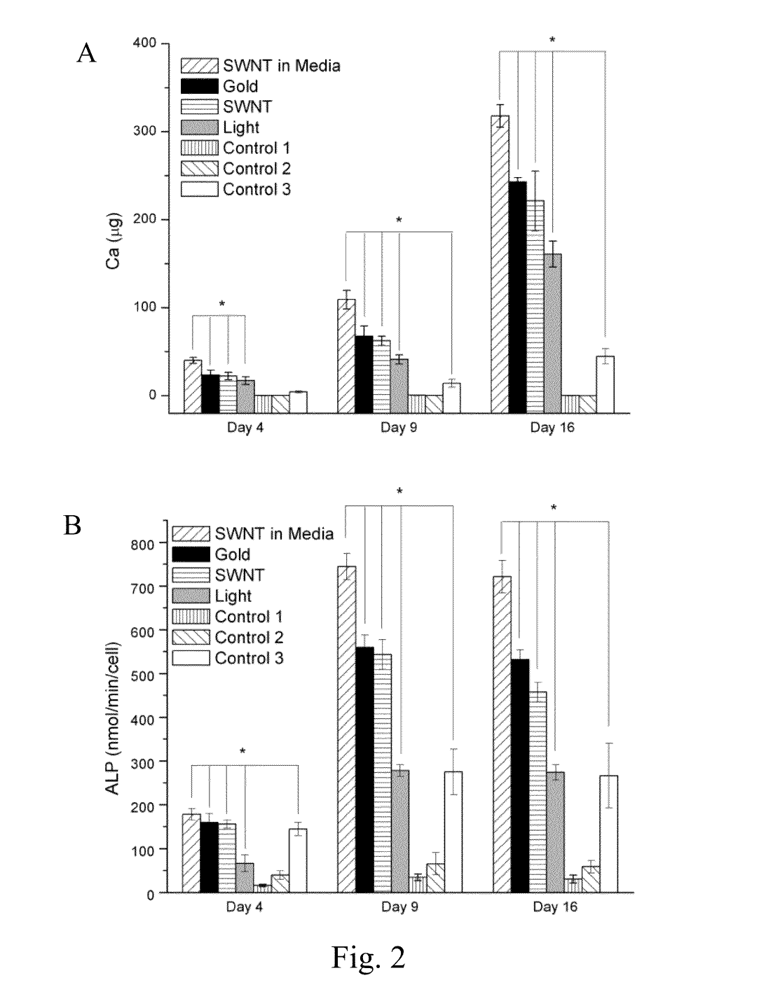

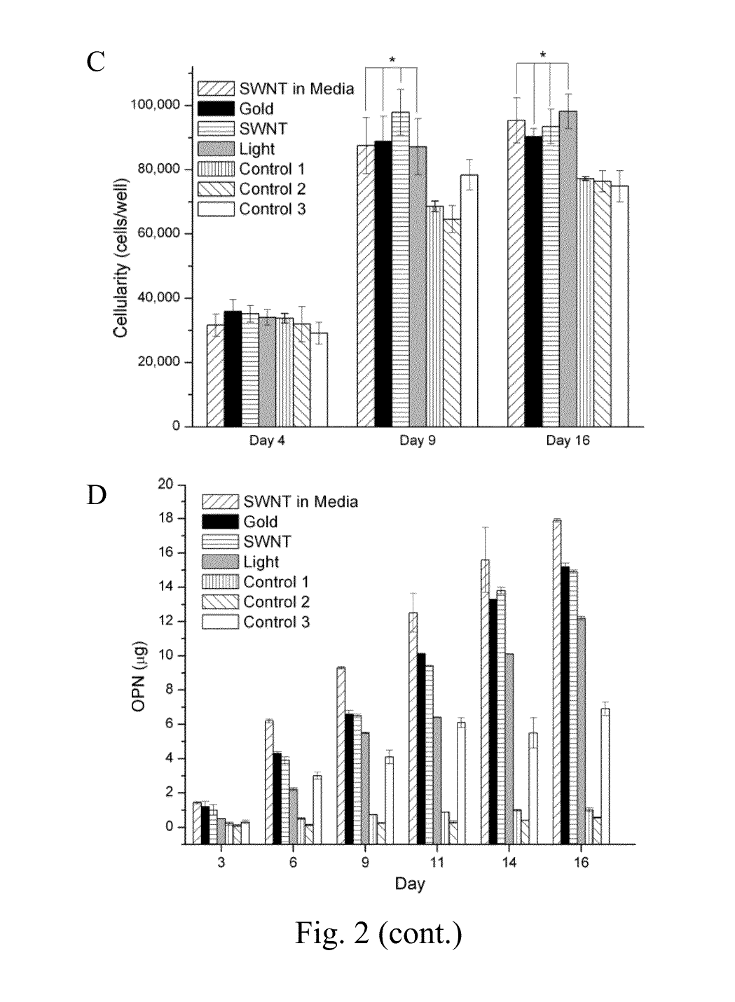

MSCs were cultured in 15 mm tissue culture plates. SWNTs or GNPs were added at 10 PPM directly to the culture. To resolve the contribution of mechanical acoustic waves generated from SWNTs or GNPs to differentiation of MSCs, SWNTs and GNPs were placed on a slide underneath the cell culture plates, avoiding direct contact with the cells. A control culture of MSCs was stimulated by laser in the absence of nanoparticles. Each culture was stimulated for 10 minutes per day by a 532 nm Nd:Yag laser with a 10 mJ pulse energy, 200 nanosecond pulse duration, 10 Hz repetition rate, and a duration of 4, 9, or 16 days. The differentiation of MSCs for the stimulated culture and a non stimulated control culture was determined by analysis of known indicators of the osteoblast phenotype including cell proliferation, production of alkaline phosphatase (ALP), deposition of a calcified matrix, and osteopontin (OPN) expression (FIG. 2A-D, FIG. 3). After 16 days, it was evident that photoacoustic stimulation increased cellular proliferation by >17%, and that osteoblast differentiation was greatly increased as determined by the calcium and ALP levels. The high level of calcium shown at day 16 implies that a mineralized bone matrix formed. ALP though is only present in the nodules of postproliferative cells, and since the Picogreen sDNA does not show a significant increase in cellularity between the 9^{th} and 16^{th} day of stimulation, it can be assumed that MSCs completed their proliferation process around the 9^{th} day of stimulation, when the level of ALP was maximum. It is likely that the DNA quantification is much higher than indicated because DNA strands may have been trapped in the extracellular matrix even after lysing the cells. At this maximum level, ALP content for SWNT and GNP groups was about 1500% greater and the light irradiated group was about 700% greater than the non stimulated control, indicating that the photoacoustic stimuli facilitated an osteoblastic lineage.

A hydrophone transducer confirmed that an acoustic signal was generated. Control cells cultured on plates with attached SWNTs or GNPs and not exposed to light indicated that acoustic energy was the greatest cause of MSC differentiation towards osteoblasts. There was no statistically significant difference between the SWNT and GNP samples which cam be accounted for by their similar resonance properties. The samples exposed directly to light had less calcium and ALP expression potentially due to the dissipation of the acoustic waves since the absorbing surface was the cellular layer.

Sulfated glycosaminoglycan (sGAG) content released into the cellular media was quantified because sGAG represents the amount of proteoglycans released from the cartilage, and we quantified the adipocyte content using Oil Red O to show that the overall trend of MSC differentiation was towards osteoblasts rather than chondrocytes and adipocytes. The sGAG and adipocyte amounts were negligible in all irradiated samples and the non irradiated control. Samples stained with Alizarin Red indicated that matrix mineralization is plentiful for stimulated cell cultures containing SWNTs in the media, as well as those outside the media.

### Example 3

### Indirect Stimulation

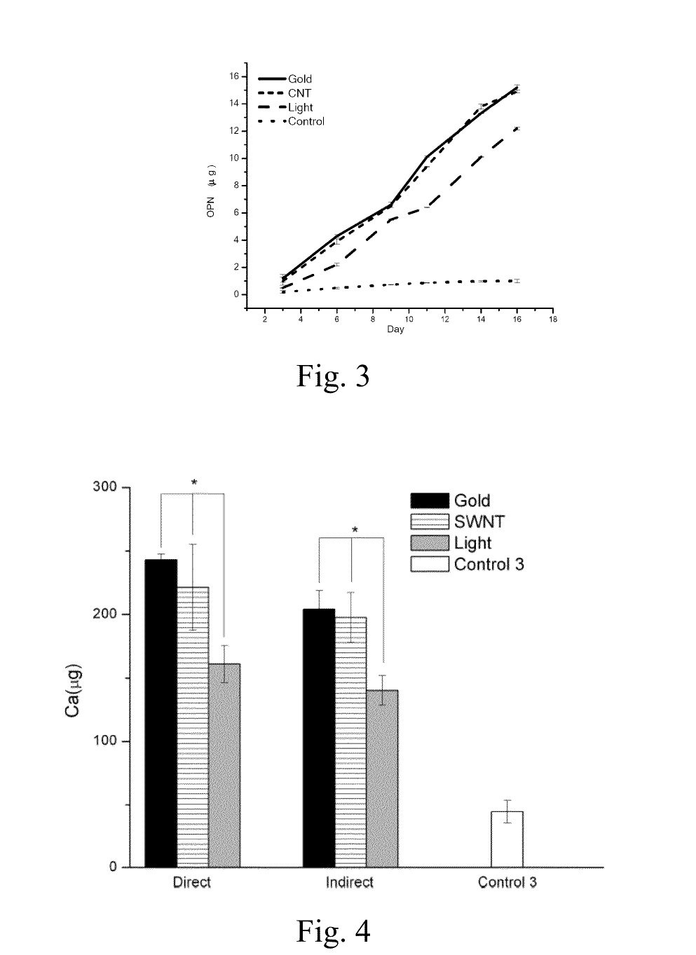

The Ca content at day 16 was compared for cells that were stimulated directly or indirectly. The cells were cultured in wells coated with SWNTs, coated with gold nanoparticles, or uncoated. Wells on which the laser impinged were considered directly exposed, while adjacent cells, which were not exposed directly to the laser pulse were considered indirectly exposed (FIG. 4). No statistically significant difference in the Ca content was observed for wells directly in the pathway of the laser light or the adjacent wells. These results indicate that for SWNT, gold nanoparticles, and pulsed laser light alone, the observed effect is mainly due to the acoustic waves. However, the greater Ca content for SWNT (˜221 μg) compared to light (˜161 μg) suggests that the acoustic waves generated by SWNTs have a greater beneficial effect on the cells.

### Example 4

### Tissue Engineering

Mouse marrow stromal cells (MSCs; ATCC-CRL12424) were used. All groups had a sample size of n=4 and are described in Table 1. For the experimental and baseline control groups, MSCs were incubated in standard DMEM (Dulbecco's Modified Eagle Medium) media, and cultured in the following three ways: 1) on bare glass cover slip; 2) on poly(lactic-co-glycolic acid) PLGA polymer film; and 3) on a PLGA-SWNT composite film. The experimental groups were: Light—MSCs cultured on glass cover slips (no polymer film) and undergoing photoacoustic (PA) stimulation; PLGA—MSCs cultured on PLGA polymer film, and undergoing PA stimulation; and PLGA-SWNT—MSCs cultured on a PLGA-SWNT composite film and undergoing PA stimulation.

TABLE 1
Experimental Groups

| | | PLGA | | Osteogenic | Photoacoustic |
|---|---|---|---|---|---|
| | Group | Film | SWNTs | Media | Stimulation |
| 1 | Light | No | No | No | Yes |
| 2 | PLGA | Yes | No | No | Yes |
| 3 | PLGA-SWNT | Yes | Yes | No | Yes |
| 4 | Light control | No | No | No | No |
| 5 | PLGA control | Yes | No | No | No |
| 6 | PLGA-SWNT | Yes | Yes | No | No |
| | control | | | | |
| 7 | Dex | No | No | Yes | No |

The three baseline controls were MSCs cultured on glass, PLGA, and PLGA-SWNT, respectively, but not exposed to PA stimulation. The positive control (Dex), consisted of MSCs grown on glass cover slips in osteogenic supplemented media (0.01 M β-glycerophosphate, 50 mg/l ascorbic acid, 10⁻⁸ M dexamethasone).

Each osteodifferentiation of the MSCs was evaluated at 4, 9, and 15 days, using cellularity, alkaline phosphatase, and calcium assays. In addition, an osteopontin assay was performed every 2-3 days. At day 15, alizarin red staining was also performed to visually detect the presence of calcium deposition.

PLGA and PLGA-SWNT Film Fabrication

Polymer coated glass slips (PLGA films) were made by a modified version of the protocol used by Karp et al., 2003, Journal of Biomedical Materials Research, 64A:388-96. PLGA films were created both with and without SWNTs. In both cases poly(lactic-co-glycolic acid; 50:50) pellets (Sigma) were weighed and dissolved at a concentration of 73 mg/ml in chloroform by heating the solution in a sealed glass vial at 60° C. for 1 hr. For the PLGA-SWNT films, SWNTs made up 0.5% w/w of the films. The liquefied PLGA and PLGA-SWNT solutions were applied in 100 μl aliquots to 15 mm round glass cover slips. The cover slips were maintained on a hot plate at 60° C. until the chloroform evaporated and films were firm. The films were then stored at 4° C. until ready for use.

In Vitro Cell Culture

MSC's were cultured onto 10 cm tissue culture plates in standard media containing DMEM (Gibco) supplemented with 10% fetal bovine serum (FBS) and 1% penicillin/streptomycin until the cells were at least 90% confluent. All MSC's were cultured in a 37° C. incubator with 95% humidity and 5% CO₂, and handled with standard tissue culture techniques. The cells were passaged, and plated onto 15 mm round glass covers slips placed in 18 mm×18 mm square glass bottom wells (Nunc), and maintained with 1.2 ml standard media. The cells for the positive control were grown on plain glass cover slips and were given 10⁻⁸ M dexamethasone (Sigma), 10 mM β-glycerophosphate (Sigma), and 50 mg/L 1-ascorbic acid (Sigma) osteogenic supplements, which have been shown to induce differentiation. (See, Porter, R. M. et al., 2003, Journal of Cellular Biochemistry, 90:13-22; Peter, S. J. et al., 1998, Journal of Cellular Biochemistry, 71:55-62) The media in all the wells were changed every 2-3 days, and the collected media were stored at 4° C. for OPN quantification. At each time point (4, 9, and 15 days) after stimulation, round cover slips from each experimental group were washed with PBS, and moved to a fresh 18 mm×18 mm square well containing 2 mL of double distilled water per sample. The samples were stored at −20° C. until the assays were performed.

Photoacoustic Protocol

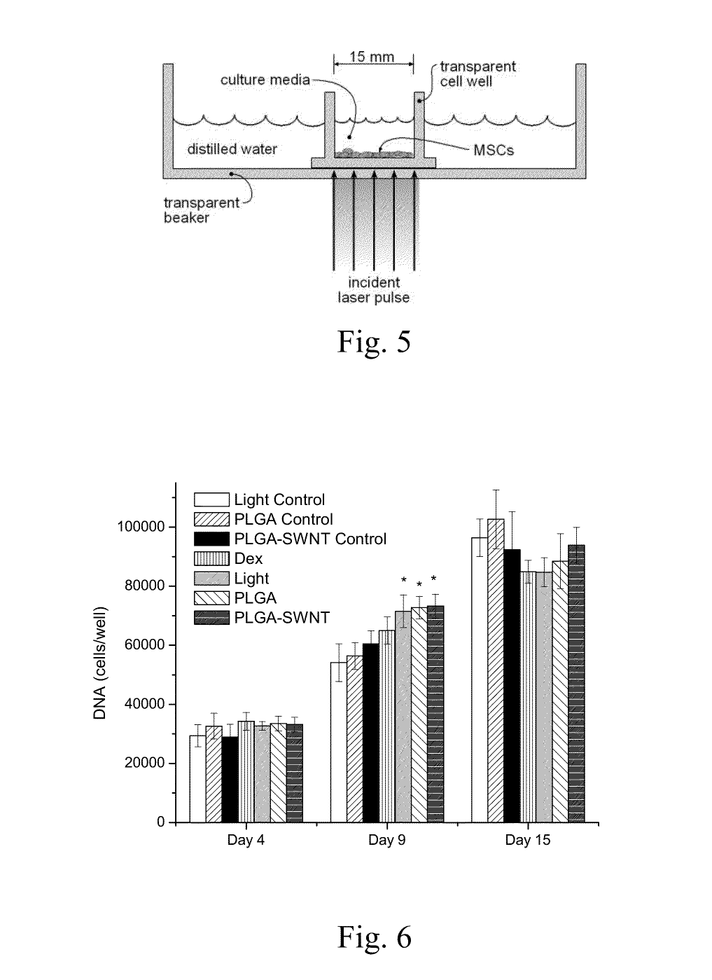

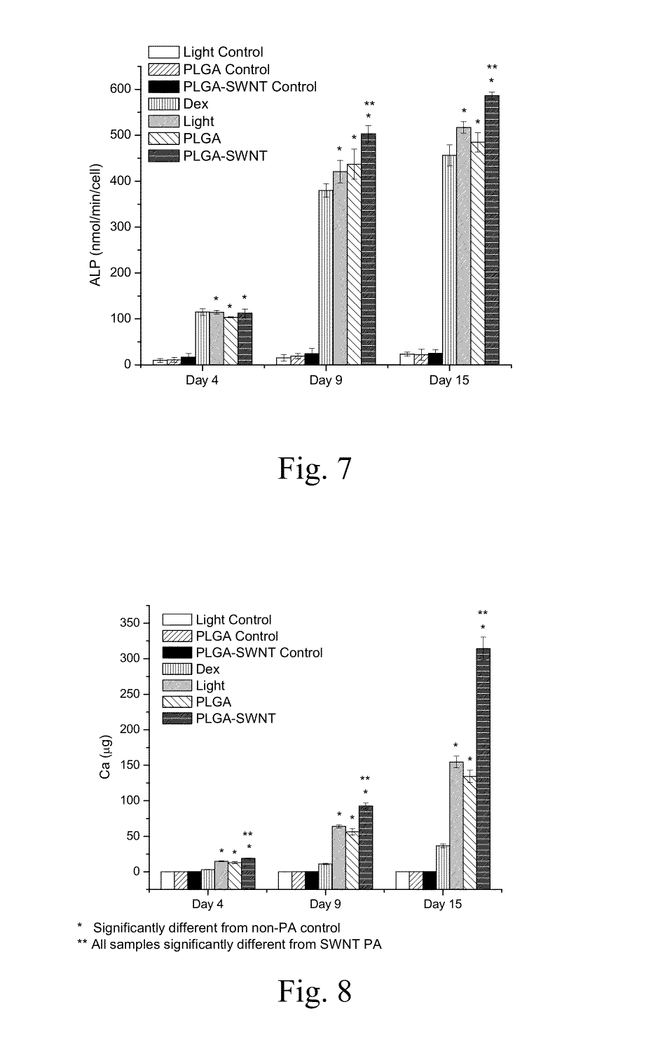

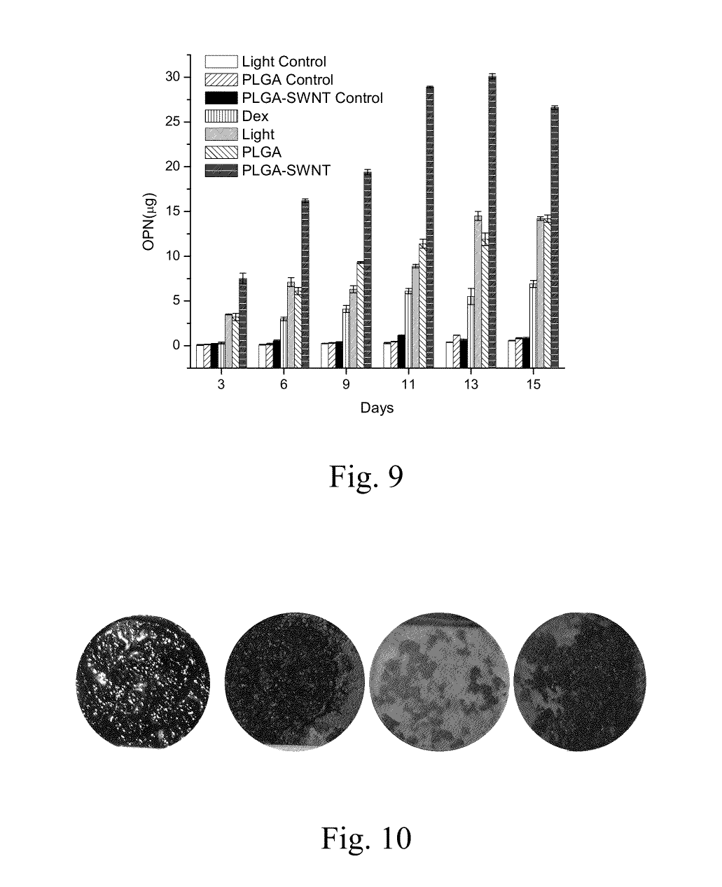

MSCs were stimulated using a 527 nm Nd:YLF short pulse laser (Photonics Industries GM-30). The laser pulses have a nominal 200 ns pulse duration, 10 mJ pulse energy, and are delivered at a rate of 10 Hz to the media. The stimulation was carried out for 10 minutes per day (˜6000 pulses/day) for 4, 9 or 15 consecutive days. The cells are held in a fixture approximately 20 cm above the optical table containing the laser. A 45° reflecting mirror below the fixture re-directs the horizontal laser beam vertically upwards, where it enters the bottom of the well. The total beam travel distance from the laser is ˜2 m, and the beam diameter is approximately 15 mm at the well bottom (FIG. 5). Control cells were maintained under similar conditions, but without photoacoustic stimulation.

To determine cell number (cellularity), DNA was quantified using a Picogreen Elisa kit (Invitrogen), which fluorescently quantifies DNA present within a sample. Quantification of cell number is possible by comparing the experimental sample DNA with the DNA in a known number of MSCs. The previously frozen cover slips containing cells were thawed and sonicated for 5 minutes to lyse the cells. A 96-well plate was then prepared with 100 μl/well of Tris EDTA buffer provided with the kit. 100 μl of standards or samples were added in triplicate to the buffer, followed by 100 μl of Picogreen reagent. The plate was incubated at room temperature in the dark for 10 minutes. The fluorescent signal was read at 480 nm excitation and 520 nm emission wavelengths using a microplate reader (Biotek, Winooski, Vt.).

The number of cells in each group was quantified over 15 days, and was found to increase over time as shown in FIG. 6. At day 9, there was a significant increase (p<0.05) in the number of cells in the PA stimulated groups versus the controls. Light, PLGA and PLGA-SWNT showed 32%, 29% and 21% more cells compared to Light Control, PLGA Control, and PLGA-SWNT Control, respectively. At day 4 no significant difference in the cell number was found between the various groups. At day 15, the cell number continued to increase but the rate of increase for the PLGA, PLGA-SWNT, and light was less between day 9 and 15 in comparison to the non-stimulated controls. Notably, between day 9 and 15, the experimental groups all became visibly confluent. Once they reach this state of confluency, they are incapable of further proliferation because there is no available surface area to allow for cellular adhesion. Accordingly, it is surmised that rapid proliferation of stimulated test cells slowed after day 9 whereas cells of the control groups, having space available, continued to proliferate.

An alkaline phosphatase assay provided a quantitative marker of early stage osteogenic activity. In a 96 well plate, 100 μl of p-Nitrophenyl Phosphate (pNPP) Liquid Substrate System (Sigma) was added to 100 μl of the sample or standard (4 nitrophenol; Sigma) in triplicate, and incubated for 1 hour at 37° C. The alkaline phosphatase produced by the cells hydrolyzes pNPP, the reagent forming p-nitrophenol. The reaction was stopped using 100 μl of 0.2 M NaOH, and absorbance.

The alkaline phosphatase assay (FIG. 7) shows a difference between the PA stimulated experimental groups and their non-stimulated controls at all three time points. At day 9, the stimulated PLGA-SWNT samples showed significantly greater (p<0.05) ALP expression than any of the other groups with 15% and 20% greater expression than PLGA and Light, respectively. By day 15, the stimulated PLGA-SWNT group showed 21% higher expression than stimulated PLGA and 13% higher expression than the Light control. The increase in ALP production from day 9 to 15 is less substantial than day 4 to 9 results. For instance, between 4 and 9 days, ALP activity for PLGA-SWNT increased by 347% whereas between 9 and 15 days, ALP expression only increased an additional 74%. The positive control Dex maintained higher ALP expression than the non-stimulated control groups at all time points, but was less than the PA stimulated groups on day 9 and 15.

Alkaline phosphatase activity for the PA stimulated groups was always statistically greater (p<0.05) than their non-stimulated controls, and the Dex group also surpassed the non-stimulated controls. This is consistent with increased ALP expression before matrix maturation. The addition of SWNTs into the PLGA matrix significantly increased ALP expression by day 9 of stimulation. Alkaline phosphatase is secreted by osteoblasts during the matrix maturation stage, making it an early-stage marker for osteogenesis. ALP expression typically stabilizes or decreases before complete matrix deposition. Thus, ALP activity may increase only slightly or not at all during later stages of differentiation. In this example, ALP activity increased at a slower rate from day 9 through day 15 as compared to day 4 through day 9.

The presence of calcium provides a later stage marker for osteogenesis, and quantifies the formation of a calcified extracellular matrix. The samples for this assay were prepared by adding 1 M acetic acid to an equal volume of the solution in each well and left on a shaker overnight to digest the biological components and dissolve the calcium into solution. Using calcium chloride as a standard, 20 μl of either standard or sample was added in triplicate to a 96 well plate. 280 μl of Arsenazo III Calcium Assay Reagent (Diagnostic Chemicals, Oxford, Conn.) was then added to each of the wells. The reagent is a calcium binding chelate, which changes color when the dissolved calcium in the sample is chelated. Absorbance was measured at 650 nm on a microplate reader (Biotek, Winooski, Vt.).

The results of the calcium assay, indicative of calcium matrix deposition, are shown in FIG. 8. Calcium matrix deposition is a late-stage marker for osteogenic differentiation. All the stimulated samples showed a temporal increase in calcium content, whereas the non-stimulated controls, with the exception of Dex, had negligible levels of calcium throughout the experiment. After 4 days of PA stimulation, PLGA-SWNT displayed a 47% and 28% greater amount of calcium than PLGA and Light respectively. This result was increased to 65% and 45% at day 9, and through day 15, where PLGA-SWNT samples had a 134%, 103%, and 760% greater calcium expression than PLGA, Light, and Dex respectively. By day 9, the calcium matrix deposition began, and matured by day 15. The positive control, Dex, followed the same trend as the PA stimulated samples but had lower levels of calcium than the PA stimulated samples at all time points. The PA groups and Dex all displayed their highest calcium expression after 15 days in culture.

Osteopontin (OPN), is an early stage marker of osteogenesis and is secreted into the extracellular media. The aspirated media changed out every 2-3 days was used for this assay. A Mouse OPN Elisa kit (R&D Systems; Minneapolis, Minn.) was used to quantify OPN. The sample media was diluted 10,000 fold in DMEM and the assay was performed in duplicate. 50 μl of the samples or standards along with 50 μl of the reagent provided with the kit were added to the OPN polyclonal antibody coated wells. The plate was incubated for two hours at room temperature to allow the OPN to bind to the antibodies. The samples were then aspirated and an enzyme-linked polyclonal antibody reagent was added for two hours at room temperature. The samples were aspirated again and 100 μl of substrate reagent was added to each well and kept for 30 minutes in the dark, during which time the enzymatic reaction occurs. Hydrochloric acid (100 μl/well) stopped the reaction. OPN levels were quantified by measuring absorbance on a microplate reader (Biotek, Winooski, Vt.) at 450 nm.

The results of the osteopontin (OPN) assay are presented in FIG. 9. Over the 15 day sequence of test, the baseline control groups had low levels of OPN secretion. The positive control and PA stimulated samples continuously increased in OPN secretion over time. The PA groups were always higher than the positive control. When the PA groups were compared to their control groups after 15 days in culture, there was a 106%, 106% and 286% increase in the PA groups for Light, PLGA, and PLGA-SWNT groups, respectively. Further, within the PA groups, the PLGA-SWNT group was 87% higher than Light and PLGA by day 15. The increase in OPN secretion in PA stimulated samples was evident after the first media change, occurring on the third day of PA stimulation, and continued to have high levels through the remainder of the study.

After as little as three days of stimulation, the PA stimulated samples had already started to secrete OPN into their extracellular fluid, which continued to increase until it peaked around day 13 for the PA stimulated samples. The OPN secretion for always surpassed all other groups at all time points. The PA stimulated Light and PLGA groups also showed increased amounts of OPN expression, though not at the level of the PA stimulated PLGA-SWNT group.

Alizarin red binds to the calcium deposited in the extracellular matrix and is a marker for matrix mineralization, a precursor to the calcified matrix associated with bone. To prepare for staining, the 15 mm cover slips of the various groups were washed with PBS, and fixed with 70% ethanol on ice for one hour. The samples were washed with ddH₂O, and stained with 500 μl of 40 mM alizarin red (Sigma-Aldrich) solution (pH 4.2) for 10 minutes at room temperature. The alizarin red solution was aspirated, and the wells were washed with ddH₂O. The samples were incubated with PBS (with no Mg or Ca) for 15 minutes at room temperature, and optical images were taken.

FIG. 10 shows representative optical images of PA stimulated PLGA-SWNT, PA stimulated PLGA, Dex(osteogenic control), and PA stimulated direct light. The deep color for the PA stimulated samples indicates the formation of a calcified matrix, which is less intense for the positive control group containing dexamethasone. The purple color in the Dex sample represents the underlying cells. These results are consistent with the quantitative calcium data, which show Dex deposits an extracellular matrix, but the level of deposition for the PA stimulated groups surpasses the non-stimulated Dex group. Although there appears to be a purple color present for the Light group, this occurs because the matrix for these samples was delicate and started to break off during the ddH₂O washing process. The matrix present on the PLGA and PLGA-SWNT samples were less delicate, so they did not experience this problem.

### REFERENCES

1. Rubin C, Turner S, Bain S, Mallinckrodt C, McLeod, K. Nature, 412, 6847, 603-604, 2001.
2. Rubin C, Capilla E, Luu Y, Busa B, Crawford H, Nolan D, Mitta V, Rosen C, Pessin J, Judex S. PNAS, 104, 45, 17879-17884, 2007.
3. Chang K, Chang W H-S. Bioelectromagnetics, 24, 3, 189-98, 2003.
4. De Pedro J A, Pérez-Caballer A J, Dominguez J, Collia F, Blanco J, Salvado M. Bioelectromagnetics, 26, 1, 20-7, 2005.
5. Sitharaman B, Shi X, Meijer G, Liao H, Walboomers F, Jansen J, Wilson L, Mikos A. Bone, 42, 2, 362-370, 2008.
6. Peng, H. B., Chang, C. W., Aloni, S., Yuzvinsky, T. D. and Zettl, A., Phys. Rev. Lett. 97, 087203, 2006.

1-52. (canceled)

53. A method of stimulating tissue growth or regeneration in a subject, comprising providing and subjecting nanoparticles to electromagnetic radiation, such that acoustic waves are induced and transmitted to the tissue.

54. The method of claim 53, wherein the tissue is bone and comprises osteocyte progenitor cells.

55. The method of claim 53, wherein the tissue is cartilage and comprises chondrocyte progenitor cells.

56. The method of claim 53, wherein the tissue is nervous tissue and comprises neural progenitor cells.

57. The method of claim 53, wherein the tissue is muscle tissue and comprises muscle progenitor cells.

58. The method of claim 53, wherein the nanoparticles are provided in a matrix.

59. The method of claim 58, wherein the tissue is bone and comprises chondrocyte progenitor cells.

60. The method of claim 58, wherein the tissue is cartilage and comprises chondrocyte progenitor cells.
